(19) United States
(12) Patent Application Publication
(10) Pub. No.: US 2003/0165546 A1
(43) Pub. Date: Sep. 4, 2003

(54) Stable personal care compositions containing a retinoid

(75) Inventors: Bradley Steven Resch, Cincinnati, OH (US); Joseph Michael Zukowski, Cincinnati, OH (US); Margaret Ann O'Donoghue, Monroe, OH (US); Shane Christian Smith, Maineville, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: THE PROCTER & GAMBLE COMPANY

(21) Appl. No.: 10/090,517

(22) Filed: Mar. 4, 2002

(51) Int. Cl. A61K 31/22; A61K 31/203; A01N 31/04; A61K 31/11

(52) U.S. Cl. 424/401; 514/546; 514/559; 514/725; 514/693

(57) ABSTRACT

The present invention relates to topical skin care compositions having improved stability containing a retinoid and a non-paraben preservative and being substantially free of paraben preservatives. Preferred non-paraben preservatives include phenols, phenol salts, carboxylic acids, carboxylic acid salts, quaternium ammonium compounds, halogens, halogen salts, alcohols, inorganic salts, heterocyclic compounds, emulsifying preservatives, and mixtures thereof. The present invention also relates to methods of using such compositions to regulate the condition of skin and/or hair.

### FIELD OF THE INVENTION

The present invention relates to the field of topical personal care compositions containing a retinoid and a preservative and to methods of use thereof.

### BACKGROUND OF THE INVENTION

Many personal care products currently available to consumers are directed primarily to improving the health and/or physical appearance of the skin and/or hair. Among the personal care products, many are directed to delaying, minimizing or even eliminating skin wrinkling and other histological changes typically associated with the aging of skin or environmental damage to human skin. Numerous compounds have been described in the art as being useful for regulating skin condition, including regulating fine lines, wrinkles and other forms of uneven or rough surface texture associated with aged or photodamaged skin.

Skin is subject to insults by many extrinsic and intrinsic factors. Extrinsic factors include ultraviolet radiation (e.g., from sun exposure), environmental pollution, wind, heat, low humidity, harsh surfactants, abrasives, and the like. Intrinsic factors include chronological aging and other biochemical changes from within the skin. Whether extrinsic or intrinsic, these factors result in visible signs of skin aging and environmental damage, such as wrinkling and other forms of roughness (including increased pore size, flaking and skin lines), and other histological changes associated with skin aging or damage. To many people, skin wrinkles are a reminder of the disappearance of youth. As a result, the elimination of wrinkles has become a booming business in youth-conscious societies. Treatments range from cosmetic creams and moisturizers to various forms of cosmetic surgery.

There are many products available to consumers designed to improve the appearance of skin. Vitamins and vitamin derivatives are commonly used in personal care products such as lotions and creams, in order to provide improved skin appearance.

One example of a vitamin that has been used to provide skin benefits in topical compositions is vitamin A, or retinol. Derivatives of vitamin A, such as esters, aldehydes, and retinoic acid, are also known to provide skin benefits. Vitamin A, along with its derivatives, form a class of compounds commonly referred to as “retinoids.” Retinoids have been found to provide a variety of skin benefits. At one time, retinoids were primarily used for the treatment of acne. More recently, retinoids have also been promoted as useful in the treatment of aged skin. Retinoids, especially retinol and retinoic acid, are known to cause skin irritation in some users. Many references are available that purport to reduce the irritation caused by retinoids.

Unfortunately, like many other vitamins and vitamin derivatives, retinoids are often reactive and susceptible to degradation, leading to a short product shelf-life and consequently the potential for consumer dissatisfaction. Some widely known sources of degradation include oxidation, light exposure, and heat. Further, there is extensive literature debating whether one retinoid is more stable than another retinoid. (For example, See, Vademecum for Vitamin age Formulations, Volker Bühler, 1988, pp 95-98). However, all sources generally agree that there is no truly stable form of retinoid, such that additional processing steps and/or packaging constraints are often required in order to minimize degradation. Degradation caused by light and heat can be easily avoided through careful handling, processing, and packaging. Degradation caused by oxidation may be at least partially alleviated by careful processing in an oxygen-free environment and oxygen impermeable packaging. However, special packaging and processing steps are not always practical or economical. Degradation may also be slowed by the inclusion of one or more anti-oxidants and/or chelating agents being added to the composition.

An added concern in personal care compositions, including those containing vitamins such as retinoids, is the potential for microorganism growth within the composition. Bacteria, yeast, and fungi may all grow in personal care compositions. If products are not appropriately protected against these organisms, product discoloration or poor product odor may result. Furthermore, the growth of some microorganisms may even contaminate the product, rendering the product dangerous for human use. Therefore there is also a need to protect retinoid compositions against the growth of microorganisms. For this reason, nearly every personal care product currently marketed contains at least one preservative to impede or eliminate the growth of unwanted organisms that may contaminate the product under normal use conditions.

Known in the art are a variety of preservatives available for use in personal care products. However, selecting one particular preservative is often complicated by several factors. For example: each preservative has a limited scope of efficacy in a given composition; some preservatives may deteriorate product quality; and many preservatives are harmful to humans when used in high dosages. Preservative selection is further complicated when formulating globally marketed products since there are very few preservatives that enjoy approval by the local regulatory agencies of each country worldwide.

Based on these and other similar selection factors, one of the most popular preservative classes currently used in personal care products is the class of preservatives known as parabens (parahydroxybenzoic acid esters). The most commonly used parabens include methylparaben, ethylparaben, propylparaben, and butylparaben. Others include isopropylparaben, isobutylparaben, and benzylparaben. Parabens are popular because they have a wide spectrum of anti-microbial activity and have few global regulatory restrictions for use. Most products that contain parabens contain a combination of different chain length esters and/or enhancers such as phenoxyethanol or benzyl alcohol.

However, it has surprisingly been discovered that retinoids may rapidly degrade when used in personal care compositions containing parabens. Therefore, there is a continuing need to formulate personal care compositions having improved stability. There is also a continuing need to formulate stable compositions which reduce the skin irritation caused by retinoids.

None of the existing art provides all of the advantages and benefits of the present invention.

### SUMMARY OF THE INVENTION

It has now surprisingly been discovered that retinoid stability may be improved in skin care compositions greatly limiting the use of parabens in the formulation. Similarly, it has been discovered that other preservatives do not cause instability when used with retinoids.

The present invention relates to topical personal care compositions having improved stability of a retinoid containing:

a) a retinoid;

b) a preservative selected from phenols, phenol salts, quaternium ammonium compounds, halogens, halogen salts, alcohols, inorganic salts, heterocyclic compounds, emulsifying preservatives, and mixtures thereof; and

c) a dermatologically acceptable carrier;

d) wherein the composition is substantially free of parahydroxybenzoic acid esters.

The present invention also relates to topical personal care compositions having improved stability of a retinoid, comprising:

a) a retinoid selected from the group consisting of retinyl esters, retinyl aldehydes, and mixtures thereof;

b) a preservative; and

c) a dermatologically acceptable carrier;

d) wherein the composition is substantially free of parahydroxybenzoic acid esters; and

e) wherein the composition is substantially free of formaldehyde and formaldehyde donating compounds.

The present invention further relates to methods of using such compositions to regulate the condition of mammalian skin and/or hair. Said methods generally contain the step of topically applying a safe and effective amount of the composition to the skin and/or hair of a mammal needing such treatment.

These and other features, aspects, and advantages of the present invention will become evident to those skilled in the art from a reading of the present disclosure.

All documents cited are, in relevant part, incorporated herein by reference; the citation of any document is not to be construed as an admission that it is prior art with respect to the present invention.

### DETAILED DESCRIPTION OF THE INVENTION

While the specification concludes with the claims particularly pointing and distinctly claiming the invention, it is believed that the present invention will be better understood from the following description.

All percentages and ratios used herein are by weight of the total composition and all measurements made are at 25° C., unless otherwise designated.

The term “ambient conditions” as used herein refers to surrounding conditions under about one atmosphere of pressure, at about 50% relative humidity, and at about 25° C. unless otherwise specified.

The compositions of the present invention can include, consist essentially of, or consist of, the components of the present invention as well as other ingredients described herein. As used herein, “consisting essentially of” means that the composition or component may include additional ingredients, but only if the additional ingredients do not materially alter the basic and novel characteristics of the claimed compositions or methods.

All percentages, parts and ratios are based upon the total weight of the personal care compositions of the present invention, unless otherwise specified. All such weights as they pertain to listed ingredients are based on the active level and, therefore, do not include carriers or by-products that may be included in commercially available materials, unless otherwise specified.

The term “dermatologically-acceptable,” as used herein, means that the compositions or components thereof so described are suitable for use in contact with mammalian keratinous tissue without undue toxicity, incompatibility, instability, allergic response, and the like.

The term “safe and effective amount” as used herein means an amount of a compound or composition sufficient to significantly induce a positive benefit, preferably a positive keratinous tissue appearance or feel benefit, or positive hair appearance or feel benefit, including independently or in combinations the benefits disclosed herein, but low enough to avoid serious side effects, i.e., to provide a reasonable benefit to risk ratio, within the scope of sound judgment of the skilled artisan.

“Signs of skin aging” include, but are not limited to, all outward visibly and tactilely perceptible manifestations as well as any other macro or micro effects due to skin aging. Such signs may be induced or caused by intrinsic factors or extrinsic factors, e.g., chronological aging and/or environmental damage. These signs may result from processes which include, but are not limited to, the development of textural discontinuities such as wrinkles and coarse deep wrinkles, skin lines, crevices, bumps, large pores (e.g., associated with adnexal structures such as sweat gland ducts, sebaceous glands, or hair follicles), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including undereye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues, especially those proximate to the skin.

It is desirable to have one or more preservatives in personal care compositions containing retinoids to improve stability of the overall product. However, it has unexpectedly been discovered that retinoids may be degraded when used in compositions containing paraben preservatives.

The use of other non-paraben preservatives in retinoid compositions, in the absence (or near absence) of paraben preservatives, has surprisingly been found to increase stability of the overall retinoid composition.

As used herein, “retinoid containing personal care products” refers to any personal care product that contains a retinoid. Preferred personal care products include products used for regulating the condition of skin, even more preferably reducing the appearance of skin aging and/or reducing the appearance or occurrence of skin acne.

The compositions of the present invention may also provide additional benefits, including absence of significant (consumer-unacceptable) skin irritation and good aesthetics.

The compositions of the present invention contain a retinoid, a preservative, and a dermatologically acceptable carrier. The compositions of the present invention are substantially free of parabens.

The compositions herein may also include a wide variety of other ingredients. The compositions of the present invention, are described in detail hereinafter.

I. Retinoid

The compositions of the present invention may contain a safe and effective amount of a retinoid. As used herein, “retinoid” includes all natural and/or synthetic analogs of Vitamin A or retinol-like compounds which possess the biological activity of Vitamin A in the skin as well as the geometric isomers and stereoisomers of these compounds. The retinoid is preferably retinol, retinol esters (e.g., C₂-C₂₂ alkyl esters of retinol, including retinyl palmitate, retinyl acetate, retinyl propionate), retinol aldehydes, retinal, beta-carotene, and/or retinoic acid (including all-trans retinoic acid and/or 13-cis-retinoic acid), more preferably retinoids other than retinoic acid. The retinoid is even more preferably a retinol ester. These compounds are well known in the art and are commercially available from a number of sources, e.g., Sigma Chemical Company (St. Louis, Mo.), Boerhinger Mannheim (Indianapolis, Ind.), BASF (Mt. Olive, N.J.), and Roche (Basel, Switzerland). Other suitable retinoids are tocopheryl-retinoate [tocopherol ester of retinoic acid (trans- or cis-), adapalene {6-[3-(1-adamantyl)-4-methoxyphenyl]-2-naphthoic acid}, and tazarotene (ethyl 6-[2-(4,4-dimethylthiochroman-6-yl)-ethynyl]nicotinate).

The retinoid may be included as the substantially pure material, or as an extract obtained by suitable physical and/or chemical isolation from natural (e.g., plant) sources. The retinoid is preferably substantially pure, more preferably essentially pure.

The compositions preferably contain from about 0.0001% to about 2%, more preferably from about 0.005% to about 2%, more preferably from about 0.01% to about 1%, more preferably from about 0.01% to about 0.5% of the retinoid. Retinol is preferably used in an amount of from about 0.01% to about 0.15%; retinol esters (e.g. retinyl propionate, retinyl acetate, retinyl palmitate) are preferably used in an amount of from about 0.01% to about 2% (e.g., about 1%); retinoic acids are preferably used in an amount of from about 0.01% to about 0.25%; tocopheryl-retinoate, adapalene, and tazarotene are preferably used in an amount of from about 0.01% to about 2%.

Mixtures of more than one retinoid may be used.

II. Preservative

The compositions of the present invention may contain a safe and effective amount of a preservative selected from: phenols and salts thereof; quaternium ammonium compounds; carboxylic acids and salts thereof; halogens and salts thereof; alcohols; inorganic salts; heterocyclic compounds; emulsifiers; and mixtures thereof. The compositions preferably contain from about 0.001% to about 1%, more preferably from about 0.001 to about 0.25%, by weight of the composition, of the preservative.

As used herein, the term “preservative,” refers to any ingredient that protects a product from the effects of microbiological contamination. Preservatives are also commonly known as anti-microbial agents or germicides. As used herein, the term “preservative” refers to any such ingredient, regardless of whether protection from microbiological contamination is the primary stated purpose of the ingredient.

Phenol Preservatives

The compositions of the present invention may contain a phenol or phenol salt preservative, collectively referred to herein as “phenol preservatives.” Phenols are synthetic or natural aromatic compounds that carry at least one —OH group on an aromatic ring. Additional ring substitutions are common.

Non-limiting examples of phenol preservatives include those listed in the table below, along with their preferred levels when present in the compositions of the present invention. Mixtures of phenol preservatives may also be incorporated:

| Phenol Preservative | Preferred Range, by weight of composition |
|---|---|
| o-phenylphenol | from about 0.05% to about 0.35% |
| sodium 0-phenylphenol | from about 0.05% to about 0.35% |
| chlorocresol | from about 0.1% to about 0.55% |
| salicylic acid | from about 0.05% to about 0.25% |
| sodium salicylate | from about 0.1% to about 1.05% |
| magnesium salicylate | from about 0.1% to about 1.05% |
| resorcin | from about 0.05% to about 0.15% |
| cresols | from about 0.001% to about 0.1% |
| chloroxylenol | from about 0.05% to about 0.25% |
| thymol | from about 0.01% to about 0.055% |
| triclosan | from about 0.01% to about 0.15% |

Quaternium Ammonium Compounds

The compositions of the present invention may contain a quaternium ammonium compound preservative. Quaternary ammonium compounds (commonly referred to in the art as “quats”) are positively charged, tetra-substituted nitrogen derivatives of the following class:

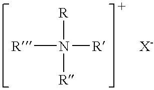

wherein R, R′, R″, and R′″ may be the same or different, but may not be hydrogen; and in which X⁻ represents a typical anion, e.g., chloride or methosulfate. If any or some of the R groups are hydrogen, the compounds of the above class are amine salts. The R groups may be aliphatic and carry additional substituents. The nitrogen atom may be part of a heterocyclic or aromatic ring system.

Non-limiting examples of quaternium ammonium compound preservatives include benzalkonium chloride, benzethonium chloride, and cetylpyridinium chloride. When present in compositions of the present invention, benzalkonium chloride is preferably included in the compositions of the present invention at a level from about 0.005% to about 0.055%, by weight of the composition; benzethonium chloride preferably from about 0.05% to about 0.25%, by weight of the composition; and cetylpyridinium chloride preferably from about 0.05% to about 1.05%, by weight of the composition.

Carboxylic Acid Preservatives

The compositions of the present invention may contain a carboxylic acid or carboxylic acid salt preservative, collectively referred to herein as “carboxylic acid preservatives.” The carboxylic acids are a group of synthetic or naturally occurring organic acids which contain at least one —COOH group.

Non-limiting examples of carboxylic acid preservatives include those listed in the table below, along with their preferred levels when present in the compositions of the present invention. Mixtures of carboxylic acid preservatives may also be incorporated:

| | Preferred |
|---|---|
| Carboxylic Acid Preservative | Range, by weight of composition |
| benzoic acid | from about 0.05% to about 0.25% |
| sodium benzoate | from about 0.05% to about 1.5% |
| sorbic acid | from about 0.05% to about 0.55% |
| dehydroacetic acid | from about 0.05% to about 0.55% |
| sodium dehydroacetate | from about 0.05% to about 1.05% |

Halogen Preservatives

The compositions of the present invention may contain a halogen or halogen salt preservative, collectively referred to herein as “halogen preservatives.” Halogens are compounds that contain one or more halogen atom(s) (Cl, Br, I, or F) covalently bonded to a carbon atom.

Non-limiting examples of halogen preservatives include those listed in the table below, along with their preferred levels when present in the compositions of the present invention. Mixtures of halogen preservatives may also be incorporated:

| | Preferred |
|---|---|
| Halogen Preservative | Range, by weight of composition |
| chlorhexidine | from about 0.01% to about 0.055% |
| chlorhexidine gluconate | from about 0.01% to about 0.055% |
| chloramine T | from about 0.05% to about 0.15% |
| triclocarban | from about 0.05% to about 0.35% |
| iodopropynyl butylcarbanate | from about 0.01% to about 0.15% |

Alcohols

The compositions of the present invention may contain an alcohol preservative. Alcohols are organic compounds in which a hydroxyl group (—OH) is attached to a saturated carbon atom. Alcohols have the general formula ROH, where R may be aliphatic or alicyclic and may include aromatic rings.

Non-limiting examples of alcohol preservatives useful herein include chlorobutanol, chlorphenesin, pentylene glycol, hinokitol, and mixtures thereof. When present in the compositions of the present invention, chlorobutanol is preferably included at a level of from about 0.01% to about 0.15%; chlorphenesin preferably from about 0.01% to about 0.35%; pentylene glycol is preferably from about 1.5% to about 10%, and hinokitol preferably from about 0.01% to about 0.15%. When present in the compositions of the present invention, pentylene glycol is most preferably included at a level of from about 2.5% to about 5%, by weight of the composition.

Inorganic Salts

The compositions of the present invention may contain an inorganic salt preservative, preferably the compositions contain from about 0.1% to about 1.05%. Inorganic Salts are the compounds formed when an inorganic base reacts with an inorganic acid. Under these circumstances, the base provides the cation while the anion is derived from the acid.

Non-limiting examples of inorganic salt preservatives useful herein include silver chloride (commercially available under the tradename Jmac), and the commercially available compound ZEOMIC.

Heterocyclic Compounds

The compositions of the present invention may contain a heterocylic compound preservative. Heterocyclic Compounds are aromatic or alicyclic compounds in which the ring contains one or more atoms other than carbon. The most common elements found in heterocyclics are nitrogen, oxygen, or sulfur. Multiple replacement of carbon in rings is not uncommon.

Non-limiting examples of heterocyclic compound preservatives useful herein include zinc pyrithione and DMDM Hydantoin. When present in compositions of the present invention, zinc pyrithione is preferably included in the compositions of the present invention at a level from about 0.001% to about 0.015%, by weight of the composition and DMDM Hydantoin preferably from about 0.01% to about 0.25%, by weight of the composition.

Emulsifier Preservatives

The compositions of the present invention may contain an emulsifier preservative. When present in compositions of the present invention, the compositions preferably contain from about 0.01% to about 0.25%, by weight of the composition, of emulsifier preservative. Emulsifier preservatives may also act as emulsifiers or surfactants in an emulsion composition, or may be combined with other emulsifiers or surfactants to form an emulsion.

Non-limiting examples of emulsifier preservatives include glycerol caprylate (commercially available as LEXGARD GMCY), alkyldiaminothylgycine hydrochloride (commercially available as TEGOQUINT), and mixtures thereof.

Mixtures of preservatives may also be used.

Preservative Enhancers

In addition to the preservatives disclosed above, the compositions of the present invention may contain a preservative enhancer. Preservative enhancers function to enhance the preservative capability of the primary preservative.

Non-limiting examples of preservative enhancers useful herein include glycols (e.g. propylene glycol, butylene glycol), EDTA and salts thereof (e.g. disodium EDTA, tetrasodium EDTA), and mixtures thereof. When present in the composition, EDTA and salts thereof are preferably used in an amount of from about 0.01% to about 0.5%, more preferably from about 0.05% to about 0.2%, by weight of the composition. When present in the composition, glycols are preferably used in an amount of from about 0.1 to about 5%, more preferably from about 0.5% to about 2%, by weight of the composition.

III. Substantially Free of Parahydroxybenzoic Acid Esters

The compositions of the present invention are substantially free of parahydroxybenzoic acids esters (i.e. “parabens”). This class of preservatives known as parabens includes methylparaben, ethylparaben, propylparaben, butylparaben, isopropylparaben, isobutylparaben, and benzylparaben.

Many raw materials commonly used in personal care compositions are sold in combination with paraben preservatives. Therefore it is difficult, if not nearly impossible, to have a personal care composition that is completely free of parabens. However, as will be understood to one of ordinary skill, it is possible to refrain from adding additional parabens to the composition, thereby maintaining a low level of parabens. As used herein, “substantially free” of parabens means that the compositions of the present invention contains less than or equal to about 0.1%, by weight of the composition, of parabens. Preferably, the compositions of the present invention contain less than about 0.01%, more preferably contain no detectable percentage, of parabens.

IV. Substantially Free of Formaldehyde and Formaldehyde-Donors

The compositions of the present invention may be substantially free of formaldehyde and substantially free of formaldehyde donors. Formaldehyde is an aldehyde that conforms to the general formula CH₂═O. There are many preservatives which act by gradually releasing formaldehyde. The term “formaldehyde donor” refers to preservatives in this class. Non-limiting examples of formaldehyde donors include dimethylol dimethylhydantoin (DMDM Hydantoin), Monomethylol dimethyl hydantoin (MDM Hydantoin), 7-(cis-3-Chloro-2-propenyl)-1,3,5-triaza-7-azoniatricyclo[3.3.1.1]decane (Dowicil 200), imidazolidinyl urea (Germall 115), and captan.

Many raw materials commonly used in personal care compositions contain small amounts of free formaldehyde. Therefore it is difficult, if not nearly impossible, to have a personal care composition that is completely free of formaldehyde and completely free of formaldehyde donors. However, as will be understood to one of ordinary skill, it is possible to refrain from adding additional formaldehyde and/or formaldehyde donors to the composition, thereby maintaining a low level of such materials. As used herein, “substantially free” of formaldehyde means that the composition contains less than 0.001%, by weight of the composition, free formaldehyde, more preferably less than 0.0002% free formaldehyde. As used herein, “substantially free” of formaldehyde donors means that the composition contains less than 0.5%, by weight of the composition, more preferably less than 0.1%, of formaldehyde donors. Preferably, the compositions of the present invention contain no detectable percentage, of total formaldehyde and no detectable percentage of formaldehyde donors.

V. Dermatologically Acceptable Carrier

The compositions of the present invention may contain a safe and effective amount of a dermatologically acceptable carrier within which other components in the composition are incorporated in order to enable the other components to be delivered to the skin at an appropriate concentration. The carrier can thus act as a diluent, dispersant, solvent, or the like for the particulate material which ensures that it can be applied to and distributed evenly over the selected target at an appropriate concentration.

The carrier may contain one or more dermatologically acceptable solid, semi-solid or liquid fillers, diluents, solvents, extenders and the like. The carrier may be solid, semi-solid or liquid. The carrier can itself be inert or it can possess dermatological benefits of its own.

Concentrations of the carrier can vary with the carrier selected and the intended concentrations of the composition components.

The type of carrier utilized in the present invention depends on the type of product form desired for the composition. The topical compositions useful in the subject invention may be made into a wide variety of product forms such as are known in the art. These include, but are not limited to, lotions, creams, gels, sticks, sprays, ointments, pastes, mousses and cosmetics (e.g., solid, semi-solid, or liquid make-up, including foundations, eye-makeup, pigmented or non-pigmented lip treatments, e.g., lipsticks, and the like). These product forms may comprise several types of carriers including, but not limited to, solutions, aerosols, emulsions, gels, solids, and liposomes.

Preferred carriers contain a dermatologically acceptable, hydrophilic diluent. As used herein, “diluent” includes materials in which the particulate material can be dispersed, dissolved, or otherwise incorporated. Non-limiting examples of hydrophilic diluents are water, organic hydrophilic diluents such as lower monovalent alcohols (e.g., C₁-C₄) and low molecular weight glycols and polyols, including propylene glycol, polyethylene glycol (e.g., Molecular Weight 200-600 g/mole), polypropylene glycol (e.g., Molecular Weight 425-2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof. Water is a preferred diluent. The composition preferably comprises from about 60% to about 99.99% of the hydrophilic diluent.

Preferably, the carrier is in the form of an emulsion. Emulsion carriers contain a hydrophilic phase comprising a hydrophilic component, e.g., water or other hydrophilic diluent, and a hydrophobic phase comprising a hydrophobic component, e.g., a lipid, oil or oily material.

As well known to one skilled in the art, the hydrophilic phase will be dispersed in the hydrophobic phase, or vice versa, to form respectively hydrophilic or hydrophobic dispersed and continuous phases, depending on the composition ingredients. The emulsion may be or comprise (e.g., in a triple or other multi-phase emulsion) an oil-in-water emulsion or a water-in-oil emulsion such as a water-in-silicone emulsion. Oil-in-water emulsions typically comprise from about 1% to about 50% (preferably about 1% to about 30%) of the dispersed hydrophobic phase and from about 1% to about 98% (preferably from about 40% to about 90%) of the continuous hydrophilic phase; water-in-oil emulsions typically comprise from about 1% to about 98% (preferably from about 40% to about 90%) of the dispersed hydrophilic phase and from about 1% to about 50% (preferably about 1% to about 30%) of the continuous hydrophobic phase. The emulsion may also comprise a gel network, such as described in G. M. Eccleston, Application of Emulsion Stability Theories to Mobile and Semisolid O/W Emulsions, Cosmetics & Toiletries, Vol. 101, November 1996, pp. 73-92.

The topical compositions of the present invention, including but not limited to lotions and creams, may comprise an emollient. Such compositions preferably contain from about 2% to about 50% of the emollient. Emollients are typically water-immiscible, oily or waxy materials. A wide variety of suitable emollients are known and may be used herein. Sagarin, Cosmetics, Science and Technology, 2nd Edition, Vol. 1, pp. 32-43 (1972), contains numerous examples of materials suitable as an emollient.

Compositions of this invention useful for cleansing (“cleansers”) are formulated with a suitable carrier, e.g., as described above, and preferably contain one or more dermatologically acceptable surfactants in an amount which is safe and effective for cleansing. Preferred compositions contain from about 1% to about 90%, more preferably from about 5% to about 10%, of a dermatologically acceptable surfactant. The surfactant is suitably selected from anionic, cationic, nonionic, zwitterionic, amphoteric and ampholytic surfactants, as well as mixtures of these surfactants. Examples of a broad variety of surfactants useful herein are described in McCutcheon's Detergents and Emulsifiers, North American Edition (1986), published by Allured Publishing Corporation. The cleansing compositions can optionally contain, at their art-established levels, other materials which are conventionally used in cleansing compositions.

The compositions of the present invention are preferably formulated to have a pH of 10.5 or below. The pH values of these compositions preferably range from about 2 to about 10.5, more preferably from about 3 to about 8, even more preferably from about 5 to about 8.

A preferred dermatologically acceptable carrier is in the form of an oil-in-water emulsion.

The dermatologically acceptable carrier may contain other ingredients, such as thickening agents, structuring agents, silicone elastomers, and mixtures thereof (more fully discussed below) in order to modify the viscosity and/or feel of the composition.

VI. Stability Criteria

The compositions of the present invention exhibit good stability. In order to measure the stability of a product, stability criteria can be established. Such criteria are based on the percentage of retinoid (by mass) remaining in a personal care product after a given time at a given temperature. For example, if 0.1% by mass of retinol was added to a product, and 0.087% of the retinol by mass remained after 4 weeks storage at 40° C., then such a product is said to have retained 87% of the original retinol after 4 weeks storage at 40° C.

Stability tests, such as shelf-life tests, may be used to evaluate stability of personal care products. In order to evaluate the stability of a retinoid in a given composition, the composition may be placed in a container that limits the free flow of oxygen (e.g. an aluminum tube) and stored for 12 weeks at a constant 40° C. and the percentage loss measured after an elapsed time period.

Optional Ingredients

The compositions of the present invention may contain one or more additional skin care components. In a preferred embodiment, where the composition is to be in contact with human keratinous tissue, the additional components should be suitable for application to keratinous tissue, that is, when incorporated into the composition they are suitable for use in contact with human keratinous tissue without undue toxicity, incompatibility, instability, allergic response, and the like within the scope of sound medical judgment.

The CTFA Cosmetic Ingredient Handbook, Second Edition (1992) describes a wide variety of non-limiting cosmetic and pharmaceutical ingredients commonly used in the personal care industry, which are suitable for use in the compositions of the present invention.

In any embodiment of the present invention, however, the actives useful herein can be categorized by the benefit they provide or by their postulated mode of action. However, it is to be understood that the actives useful herein can in some instances provide more than one benefit or operate via more than one mode of action. Therefore, classifications herein are made for the sake of convenience and are not intended to limit the active to that particular application or applications listed.

Silicone Elastomers

The compositions of the present invention may contain a silicone elastomer. When present, the composition preferably comprises from about 0.1% to about 30%, more preferably from about 1% to about 20%, and even more preferably, from about 2% to about 10%, by weight of the composition, of a silicone elastomer component.

The compositions of the present invention may include an emulsifying crosslinked organopolysiloxane elastomer, a non-emulsifying crosslinked organopolysiloxane elastomer, or a mixture thereof. The term “non-emulsifying,” as used herein, defines crosslinked organopolysiloxane elastomers from which polyoxyalkylene units are absent. The term “emulsifying,” as used herein, means crosslinked organopolysiloxane elastomers having at least one polyoxyalkylene (e.g., polyoxyethylene or polyoxypropylene) unit.

No specific restriction exists as to the type of curable organopolysiloxane composition which can serve as starting material for the crosslinked organopolysiloxane elastomer.

Non-limiting examples of emulsifying elastomers include polyoxyalkylene modified elastomers formed from divinyl compounds, particularly siloxane polymers with at least two free vinyl groups, reacting with Si—H linkages on a polysiloxane backbone. Preferably, the elastomers are dimethyl polysiloxanes crosslinked by Si—H sites on a molecularly spherical MQ resin. Emulsifying crosslinked organopolysiloxane elastomer can notably be chosen from the crosslinked polymers described in U.S. Pat. Nos. 5,412,004 (issued May 2, 1995); 5,837,793 (issued Nov. 17, 1998); and 5,811,487 (issued Sep. 22, 1998). In addition, an emulsifying elastomer comprised of dimethicone copolyol crosspolymer (and) dimethicone is available from Shin Etsu under the tradename KSG-21.

Non-limiting examples of non-emulsifying elastomers are dimethicone/vinyl dimethicone crosspolymers. Such dimethicone/vinyl dimethicone crosspolymers are supplied by a variety of suppliers including Dow Coming (DC 9040 and DC 9041), General Electric (SFE 839), Shin Etsu (KSG-15, 16, 18 [dimethicone/phenyl vinyl dimethicone crosspolymer]), and Grant Industries (GRANSIL™ line of elastomers). Cross-linked organopolysiloxane elastomers useful in the present invention and processes for making them are further described in U.S. Pat. No. 4,970,252 to Sakuta, et al., issued Nov. 13, 1990; U.S. Pat. No. 5,760,116 to Kilgour, et al., issued Jun. 2, 1998; U.S. Pat. No. 5,654,362 to Schulz, Jr., et al. issued Aug. 5, 1997. Additional crosslinked organopolysiloxane elastomers useful in the present invention are disclosed in Japanese Patent Application JP 61-18708, assigned to Pola Kasei Kogyo KK.

Commercially available elastomers preferred for use herein are Dow Corning's 9040 silicone elastomer blend, Shin Etsu's KSG-21, and mixtures thereof.

Structuring Agent

The compositions of the present invention, in some embodiments, may further include a structuring agent. Compositions of this invention may contain from about 0.1% to about 20%, more preferably from about 0.1% to about 10%, still more preferably from about 0.5% to about 9%, of one or more structuring agents.

Preferred structuring agents for use herein are those having an HLB of from about 1 to about 8 and having a melting point of at least about 45° C. Non-limiting examples of structuring agents useful in compositions of the present invention include stearic acid, palmitic acid, stearyl alcohol, cetyl alcohol, behenyl alcohol, stearic acid, palmitic acid, the polyethylene glycol ether of stearyl alcohol having an average of about 1 to about 5 ethylene oxide units, the polyethylene glycol ether of cetyl alcohol having an average of about 1 to about 5 ethylene oxide units, and mixtures thereof.

Thickening Agents

The compositions of the present invention, in some embodiments, may further include one or more thickening agents. When present, the composition preferably includes from about 0.1% to about 5%, more preferably from about 0.1% to about 4%, and still more preferably from about 0.25% to about 3%, by weight of the composition of the thickening agent.

Nonlimiting examples of thickening agents useful herein include carboxylic acid polymers such as the carbomers (such as those commercially available under the tradename Carbopol® 900 series from B. F. Goodrich; e.g., Carbopol® 954). Other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as Carbopol® 1342, Carbopol® 1382, PEMULEN TR-1, and PEMULEN TR-2, from B. F. Goodrich.

Other nonlimiting examples of thickening agents include crosslinked polyacrylate polymers including both cationic and nonionic polymers.

Still other nonlimiting examples of thickening agents include the polyacrylamide polymers, especially nonionic polyacrylamide polymers including substituted branched or unbranched polymers. More preferred among these polyacrylamide polymers is the nonionic polymer given the CTFA designation polyacrylamide and isoparaffin and laureth-7, available under the Tradename Sepigel 305 from Seppic Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500W, SSSA100H, from Lipo Chemicals, Inc., (Patterson, N.J.).

Another nonlimiting class of thickening agents useful herein are the polysaccharides. Nonlimiting examples of polysaccharide gelling agents include those selected from cellulose, and cellulose derivatives. Preferred among the alkyl hydroxyalkyl cellulose ethers is the material given the CTFA designation cetyl hydroxyethylcellulose, which is the ether of cetyl alcohol and hydroxyethylcellulose, sold under the tradename Natrosol® CS Plus from Aqualon Corporation (Wilmington, Del.). Other useful polysaccharides include scleroglucans which are a linear chain of (1-3) linked glucose units with a (1-6) linked glucose every three units, a commercially available example of which is Clearogel™ CS11 from Michel Mercier Products Inc. (Mountainside, N.J.).

Another nonlimiting class of thickening agents useful herein is the gums. Nonlimiting examples of gums useful herein include hectorite, hydrated silica, xantham gum, and mixtures thereof.

Vitamins

Non-limiting examples of vitamins useful herein include vitamin B₃ compounds (such as niacinamide, tocopherol nicotinate), vitamin C (such as magnesium ascorbyl phosphate, ascorbyl glucoside), Vitamin A or derivatives (such as retinol, retinyl palmitate, retinyl acetate, retinyl propionate), Vitamin B₅ or derivatives (such as panthenol, pantothenoic acid), Vitamin E or derivatives (such as tocopherol, tocopherol acetate), or Vitamin D₃ or derivatives.

Vitamin B₃ Compounds

The compositions of the present invention may include, in some embodiments, a vitamin B₃ compound. Salts of the vitamin B₃ compound are also useful herein. When present, the composition preferably includes from about 0.01% to about 50%, more preferably from about 0.1% to about 10%, by weight of the composition, of the vitamin B₃ compound.

Non-limiting examples of vitamin B₃ compounds useful herein include niacinamide, tocopherol nicotinate, and mixtures thereof.

Proteins

Non-limiting examples of proteins useful herein include hydrolyzed and non-hydrolyzed (i.e. “native”) animal and vegetable derived proteins. A particularly preferred protein is hydrolyzed wheat protein.

Non-limiting examples of sources of hydrolyzed and/or partially-hydrolyzed plant derived proteins include: soya proteins, wheat proteins, almond protein, potato protein, oat proteins, pea proteins, sun flower proteins, corn proteins, cottonseed proteins, peanut proteins, and wheat germ protein. Other non-limiting examples include commercially available compounds containing hydrolyzed vegetable protein (and) hydrolyzed vegetable starch. Non-limiting examples of hydrolyzed and/or partially-hydrolyzed animal derived proteins useful herein include: milk proteins, such as ,lactoglobulin, casein, or whey; serum proteins, such as horse serum; placental proteins; albumen; amylase; collagen; crystalline; cytochrome C; elastin; fibronectin; gelatin; gliadin; keratin; lipase; and serum albumin.

Non-limiting examples of plant derived non-hydrolyzed proteins useful herein, include: soya proteins, wheat proteins, almond protein, potato protein, oat proteins, pea proteins, sun flower proteins, corn proteins, cottonseed proteins, peanut proteins, and wheat germ protein. Non-limiting examples of animal derived non-hydrolyzed proteins useful herein, include: milk proteins, such as ,lactoglobulin, casein, or whey; serum proteins, such as horse serum; placental proteins; albumen; amylase; collagen; crystalline; cytochrome C; elastin; fibronectin; gelatin; gliadin; keratin; lipase; and serum albumin.

Zeolites

Non-limiting examples of zeolites useful herein include natural zeolites such as analcite, chabazite, heulandite, natrolite, stilbite, and thomosonite; and synthetic zeolites such as those made by the gel process (sodium silicate and alumina) or a clay process (kaolin), which forms a matrix to which the zeolite is added.

Peptides

Peptides, including but not limited to, di-, tri-, tetra-, and pentapeptides and derivatives thereof, may be included in the compositions of the present invention in amounts that are safe and effective. Non-limiting examples of peptides and peptide derivatives useful herein include; Camosine® (beta-ala-his), gly-his-lys, arg-lys-arg, his-gly-gly, palmitoyl-gly-his-lys (which may be purchased as Biopeptide CL®, 100 ppm commercially available from Sederma, France), Peptide CK (arg-lys-arg), PEPTIDE CK+ (ac-arg-lys-arg-NH₂), and a copper derivative of his-gly-gly sold commercially as IAMIN, from Sigma (St. Louis, Mo.). Tetrapeptides and pentapeptides (such as palmitoyl-lys-thr-thr-lys-ser commercially available from Sederma France) are also suitable for use herein.

When included in the present compositions, peptides are preferably included in amounts of from about 1×10⁻⁶% to about 10%, more preferably from about 1×10⁻⁶% to about 0.1%, by weight of the composition.

Sunscreen Actives

The compositions of the subject invention may contain a sunscreen active. As used herein, “sunscreen active” includes both sunscreen agents and physical sunblocks.

Inorganic sunscreens useful herein include the following metallic oxides; titanium dioxide having an average primary particle size of from about 15 nm to about 100 nm, zinc oxide having an average primary particle size of from about 15 nm to about 150 nm, iron oxide having an average primary particle size of from about 15 nm to about 500 nm, and mixtures thereof. When used herein, the inorganic sunscreens are present in the amount of from about 0.1% to about 20%, preferably from about 0.5% to about 10%, by weight of the composition.

A wide variety of conventional organic sunscreen actives are suitable for use herein. Sagarin, Vol. 102 pages 21 et seq., of Cosmetics and Toiletries (1987), discloses numerous suitable actives. Nonlimiting examples of organic sunscreen actives useful herein include octylsalicylate, 2-Phenylbenzimidazole-5-sulphonic acid salts, Salts of Terephthalylidene Dicamphor sulfonic acid, octocrylene, octylmethoxycinnamate, avobenzone, and mixtures thereof.

When present in compositions of the present invention, a safe and effective amount of the organic sunscreen active is used, typically from about 1% to about 20%, more typically from about 2% to about 10% by weight of the composition.

Terpene Alcohols

The topical compositions of the present invention may, in some embodiments, contain a safe and effective amount of a terpene alcohol such as phytantriol, phytantriol derivatives, farnesol, farnesol derivatives, and mixtures thereof. When included in compositions of the present invention, the terpene alcohol is preferably is included in an amount from about 0.001% to about 50% by weight of the composition, more preferably from about 0.01% to about 20%, by weight of the composition.

Desquamation Actives

A safe and effective amount of a desquamation active may be added to the compositions of the present invention, preferably from about 0.1% to about 10%, more preferably from about 0.2% to about 5%, by weight of the composition. Non-limiting examples of desquamation systems useful herein include; a combination of sulfhydryl compounds and zwitterionic surfactants; and a combination of salicylic acid and zwitterionic surfactants.

Anti-Acne Actives

The compositions of the present invention may contain a safe and effective amount of one or more anti-acne actives. Examples of useful anti-acne actives include resorcinol, sulfur, salicylic acid, benzoyl peroxide, erythromycin, zinc, etc.

Anti-Wrinkle Actives/Anti-Atrophy Actives

The compositions of the present invention may further contain a safe and effective amount of one or more anti-wrinkle actives or anti-atrophy actives. Non-limiting examples of anti-wrinkle/anti-atrophy actives suitable for use in the compositions of the present invention include hydroxy acids (e.g., alpha-hydroxy acids such as lactic acid and glycolic acid or beta-hydroxy acids such as salicylic acid and salicylic acid derivatives such as the octanoyl derivative), phytic acid, lipoic acid; lysophosphatidic acid, and skin peel agents.

Anti-Oxidants/Radical Scavengers

A safe and effective amount of an anti-oxidant/radical scavenger may be added to the compositions of the subject invention, preferably from about 0.1% to about 10%, more preferably from about 1% to about 5%, of the composition.

Non-limiting examples of anti-oxidants/radical scavengers useful herein include; ascorbic acid (vitamin C) and derivatives thereof; tocopherol (vitamin E) and derivatives thereof (e.g. tocopherol sorbate, tocopherol acetate); butylated hydroxy benzoic acids and their salts, 6-hydroxy-2,5,7,8-tetramethylchroman-2-carboxylic acid; sorbic acid and its salts; lipoic acid, amines (e.g., N,N-diethylhydroxylamine, amino-guanidine); tea extracts; grape skin/seed extracts; and mixtures thereof.

Flavonoids

The compositions of the present invention may optionally contain a flavonoid compound. Flavonoids are broadly disclosed in U.S. Pat. Nos. 5,686,082 and 5,686,367. Non-limiting examples of flavonoids useful herein include unsubstituted flavone, 7,2′-dihydroxy flavone, 3′,4′-dihydroxy naphthoflavone, 4′-hydroxy flavone, 5,6-benzoflavone, and 7,8-benzoflavone, unsubstituted isoflavone, daidzein (7,4′-dihydroxy isoflavone), 5,7-dihydroxy-4′-methoxy isoflavone, soy isoflavones (a mixture extracted from soy), and mixtures thereof.

When present, the flavonoid compounds are preferably present in concentrations of from about 0.01% to about 20%, more preferably from about 0.1% to about 10%, by weight of the composition.

Anti-Inflammatory Agents

A safe and effective amount of an anti-inflammatory agent may be added to the compositions of the present invention, preferably from about 0.1% to about 10%, more preferably from about 0.5% to about 5%, of the composition.

Nonlimiting examples of “natural” anti-inflammatory agents that are useful herein include candelilla wax, bisabolol (e.g., alpha bisabolol), aloe vera, plant sterols (e.g., phytosterol), and mixtures thereof.

Additional anti-inflammatory agents useful herein include glycyrrhizinate compounds such as dipotassium glycyrrhizinate.

Anti-Cellulite Agents

The compositions of the present invention may also contain a safe and effective amount of an anti-cellulite agent. Non-limiting examples of anti-cellulite agents useful herein include xanthine compounds (e.g., caffeine, theophylline, theobromine, and aminophylline).

Topical Anesthetics

The compositions of the present invention may also contain a safe and effective amount of a topical anesthetic. Examples of topical anesthetic drugs include benzocaine, lidocaine, pharmaceutically acceptable salts thereof, and mixtures thereof.

Tanning Actives

The compositions of the present invention may contain a tanning active. When present, it is preferable that the compositions contain from about 0.1% to about 20%, more preferably from about 2% to about 7%, by weight of the composition, of the artificial tanning active.

A non-limiting example of a tanning active useful herein is dihydroxyacetone.

Skin Lightening Agents

The compositions of the present invention may contain a skin lightening agent. When used, the compositions preferably contain from about 0.1% to about 10%, more preferably from about 0.2% to about 5%, by weight of the composition, of a skin lightening agent. Non-limiting examples of skin lightening agents useful herein include those known in the art, including niacinamide, kojic acid, arbutin, ascorbic acid and derivatives thereof (e.g sodium ascorbyl phosphate), and extracts (e.g., mulberry extract, placental extract).

Skin Soothing and Skin Healing Actives

The compositions of the present invention may include a skin soothing or skin healing active. Skin soothing or skin healing actives suitable for use herein include panthenoic acid derivatives (including panthenol, dexpanthenol, ethyl panthenol), aloe vera, allantoin, bisabolol, and dipotassium glycyrrhizinate. A safe and effective amount of a skin soothing or skin healing active may be added to the present composition, preferably, from about 0.1% to about 30%, more preferably from about 0.5% to about 20%, by weight of the composition.

Particulate Material

The compositions of the present invention may, in some embodiments, contain a particulate material, preferably a metallic oxide. These particulates can be coated or uncoated, charged or uncharged. Non-limiting examples of particulate materials useful herein include; iron oxide, mica, mica treated with barium sulfate, titanium dioxide (TiO2), zinc oxide, silica, nylon, polyethylene, talc, styrene, polypropylene, ethylene/acrylic acid copolymer, sericite, aluminum oxide, silicone resin, barium sulfate, polymethyl methacrylate, and mixtures thereof.

When present, particulate materials are present in a safe and effective amount, preferably in levels of from about 0.01% to about 2%, more preferably from about 0.05% to about 1.5%, still more preferably from about 0.1% to about 1%, by weight of the composition.

Conditioning Agent

Some embodiments of the present invention may further contain a conditioning agent selected from humectants, moisturizers, or skin conditioners. A variety of these materials can be employed and each can be present at a level of from about 0.01% to about 20%, more preferably from about 0.1% to about 10%, by weight of the composition. Nonlimiting examples of conditioning agents useful herein include hyaluronic acid, glycerin, panthenol, allantoin, and mixtures thereof. Also useful are various C₁-C₃₀ monoesters and polyesters of sugars and related materials.

Methods of Use

The compositions of the present invention are useful for regulating the condition of skin and/or hair while maintaining good stability. Regulating the condition of skin includes reducing the appearance of fine lines and/or wrinkles on the skin, reducing the appearance of eye bags and dark circles under the eyes, sagging skin, scars/marks, dimples, pores, stretch marks, roughness, skin surface blemishes, frown lines, expression lines, rhytides, blemishes, photodamage, crevices, and/or unevenness. Regulating the condition of skin also includes reducing the occurrence and/or appearance of acne.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Making Instructions For All Examples

All of the following examples can be made according to the following instructions:

In a suitable container, all water phase ingredients are mixed together. Then, in a separate container, all of the oil phase ingredients are mixed together. Each phase is separately heated to 75° C. When both phases reach 75° C., the oil phase is added to the water phase and the mixture is emulsified using a suitable mill (e.g. Tekmar T-25) for approximately 5 minutes. Following emulsification, the pH is adjusted to the desired level. The mixture is then cooled to 60° C. and any remaining ingredients (except for the retinoid phase) are added to the mixture with mixing. The mixture is then slowly permitted to cool. The retinoid-phase ingredients are mixed together in a separate container to form a retinoid premix. The premix is emulsified using a mill for approximately one minute. When the main mixture reaches 40° C., the retinoid premix is added to the main mixture. The batch is then cooled to 35° C. At 35° C., the batch is milled again for approximately five minutes, and the finished product is transferred to suitable containers.

Topical Cream

| | % w/w | | | |
|---|---|---|---|---|
| Ingredient | 1a | 1b | 1c | 1d |
| Phase A | | | | |
| Water | q.s. 100% | q.s. 100% | q.s. 100% | q.s. 100% |
| Glycerin | 3.0 | 3.0 | 3.0 | 3.0 |
| Sodium Citrate | 0.1 | 0.1 | 0.1 | 0.1 |
| Disodium EDTA | 0.1 | 0.1 | 0.1 | 0.1 |
| Sodium Benzoate | — | 0.2 | — | — |
| Benzalkonium | — | — | 0.2 | — |
| Chloride | | | | |
| Hinokitol | — | — | — | 0.1 |
| Dehydroacetic | — | 0.1 | — | — |
| Acid | | | | |
| Sorbic Acid | 0.1 | — | — | — |
| Chlorhexidine | — | 0.05 | 0.05 | — |
| Thymol | — | — | — | 0.1 |
| Phase B | | | | |
| Octyl | 4.0 | 4.0 | 4.0 | 4.0 |
| Hydroxystearate | | | | |
| Ceteareth-20 | 2.0 | 2.0 | 2.0 | 2.0 |
| Dimethicone | 1.0 | 1.0 | 1.0 | 1.0 |
| C12-15 Alkyl | 2.0 | 2.0 | 2.0 | 2.0 |
| Lactate | | | | |
| Steareth-10 | 1.0 | 1.0 | 1.0 | 1.0 |
| Stearyl Alcohol | 1.0 | 1.0 | 1.0 | 1.0 |
| Cholesterol | 0.25 | 0.25 | 0.25 | 0.25 |
| Cetearyl Alcohol | 0.5 | 0.5 | 0.5 | 0.5 |
| Acetylated | 0.5 | 0.5 | 0.5 | 0.5 |
| Lanolin | | | | |
| Polysorbate-80 | 0.2 | 0.2 | 0.2 | 0.2 |
| Glyceryl | 1.0 | 1.0 | 1.0 | 1.0 |
| Distearate | | | | |
| Phasae C | | | | |
| Sodium Hydroxide | (Adjust pH) | | | |
| Phase D | | | | |
| Sepigel 305 | 2.0 | 2.0 | 2.0 | 2.0 |
| Phase E | | | | |
| C12-15 Alkyl | 1.0 | 1.0 | 1.0 | 1.0 |
| Benzoate | | | | |
| Polysorbate-20 | 0.1 | 0.1 | 0.1 | 0.1 |
| Retinol | 0.1 | 0.1 | 0.1 | 0.1 |
| Water | 3.0 | 3.0 | 3.0 | 3.0 |
| BHT | 0.01 | 0.01 | 0.01 | 0.01 |
| BHA | 0.01 | 0.01 | 0.01 | 0.01 |

Topical Cream

| | % w/w | | | |
|---|---|---|---|---|
| Ingredient | 2a | 2b | 2c | 2d |
| Phase A | | | | |
| Water | q.s. 100% | q.s. 100% | q.s. 100% | q.s. 100% |
| PVM/MA Copolymer | 0.5 | 0.5 | 0.5 | 0.5 |
| Glycerin | 1.0 | 1.0 | 1.0 | 1.0 |
| Dipropylene Glycol | 1.5 | — | 1.5 | — |
| Pentylene Glycol | — | 3.5 | — | 1.5 |
| Chlorhexidine | 0.05 | — | 0.05 | 0.05 |
| Sodium Benzoate | 0.2 | — | 0.2 | 0.2 |
| Phase B | | | | |
| Caprylic/Capric | 5.0 | 5.0 | 5.0 | 5.0 |
| Triglyceride | | | | |
| Glyceryl Stearate SE | 2.0 | 2.0 | 2.0 | 2.0 |
| Squalane | 2.0 | 2.0 | 2.0 | 2.0 |
| Cetearyl Glycoside | 1.0 | 1.0 | 1.0 | 1.0 |
| Glyceryl | 0.5 | 0.5 | 0.5 | 0.5 |
| Polymethacrylate | | | | |
| Phase C | | | | |
| Sodium Hydroxide | (Adjust pH) | | | |
| Phase D | | | | |
| Sepigel 305 | 1.5 | 1.5 | 1.5 | 1.5 |
| Phase E | | | | |
| Caprylic/Capric | 1.5 | 1.5 | 1.5 | 1.5 |
| Triglyceride | | | | |
| Lecithin | 0.1 | 0.1 | 0.1 | 0.1 |
| Retinyl Propionate | 0.1 | 0.1 | — | — |
| Retinyl Acetate | — | — | 0.1 | 0.1 |
| Water | 4.0 | 4.0 | 4.0 | 4.0 |
| BHT | 0.02 | 0.02 | 0.02 | 0.02 |

Topical Cream

| | % w/w | | | |
|---|---|---|---|---|
| Ingredient | 3a | 3b | 3c | 3d |
| Phase A | | | | |
| Water | q.s. 100% | q.s. 100% | q.s. 100% | q.s. 100% |
| Glycerin | 3.0 | 3.0 | 3.0 | 3.0 |
| Phosphoric Acid | 0.1 | 0.1 | 0.1 | 0.1 |
| Chloramine T | — | 0.15 | — | — |
| Chlorbutanol | — | — | 0.1 | 0.1 |
| Sodium o- | 0.1 | — | — | 0.1 |
| phenylphenol | | | | |
| Triclosan | — | 0.2 | — | — |
| Sodium Salicylate | — | — | 0.1 | — |
| Phenol | 0.1 | — | — | — |
| Phase B | | | | |
| Squalane | 5.0 | 5.0 | 5.0 | 5.0 |
| Cetyl Alcohol | 1.0 | 1.0 | 1.0 | 1.0 |
| Glyceryl Stearate | 1.0 | 1.0 | 1.0 | 1.0 |
| PEG-40 Stearate | 1.0 | 1.0 | 1.0 | 1.0 |
| Petrolatum | 0.5 | 0.5 | 0.5 | 0.5 |
| Isohexadecane | 0.1 | 0.1 | 0.1 | 0.1 |
| Sorbitan Tristearate | 0.05 | 0.05 | 0.05 | 0.05 |
| Hydrogeante | 0.1 | 0.1 | 0.1 | 0.1 |
| Polyisobutene | | | | |
| Cholesterol | 0.01 | 0.01 | 0.01 | 0.01 |
| Paraffin | 0.01 | 0.01 | 0.01 | 0.01 |
| Phase C | | | | |
| Sodium Hydroxide | (Adjust pH) | | | |
| Phase D | | | | |
| Squalane | 2.0 | 2.0 | 2.0 | 2.0 |
| Lecithin | 0.1 | 0.1 | 0.1 | 0.1 |
| Retinol | 0.1 | 0.1 | 0.1 | 0.1 |
| Retinyl Palmitate | 0.05 | 0.05 | 0.05 | 0.05 |
| Water | 4.0 | 4.0 | 4.0 | 4.0 |
| BHA | 0.02 | 0.02 | 0.02 | 0.02 |

Topical Lotion

| | % w/w | | | |
|---|---|---|---|---|
| Ingredient | 4a | 4b | 4c | 4d |
| Phase A | | | | |
| Water | q.s. 100% | q.s. 100% | q.s. 100% | q.s. 100% |
| Xantham gum | 0.1 | 0.1 | 0.1 | 0.1 |
| Polymethyl methacrylate | 0.2 | 0.2 | 0.2 | 0.2 |
| Butylene glycol | 3.0 | 3.0 | 3.0 | 3.0 |
| Glycerin | 3.0 | 3.0 | 3.0 | 3.0 |
| Sodium polyaspartate | 0.05 | 0.05 | 0.05 | 0.05 |
| Phosphoric Acid | 0.05 | 0.05 | 0.05 | 0.05 |
| Sucrose | 0.5 | 0.5 | 0.5 | 0.5 |
| Potasium Sulfate | 0.2 | 0.2 | 0.2 | 0.2 |
| Resorcinol | — | 0.1 | — | — |
| Dehydroacetic Acid | 0.1 | — | — | — |
| Chlorhexidine Gluconate | — | — | 0.05 | 0.05 |
| Glycerol Caprylate | — | — | 0.1 | — |
| Sodium Benzoate | 0.2 | — | — | 0.2 |
| Phase B | | | | |
| cyclomethicone | 7.0 | 7.0 | 7.0 | 7.0 |
| Polymethylsilsequioxane | 3.0 | 3.0 | 3.0 | 3.0 |
| caprylic/cparic/stearic | 3.0 | 3.0 | 3.0 | 3.0 |
| triglyceride | | | | |
| Dimethicone copolyol | 1.0 | 1.0 | 1.0 | 1.0 |
| hydrogenated | 0.2 | 0.2 | 0.2 | 0.2 |
| polyisobutene | | | | |
| PEG-60 Hydrogenate | 0.5 | 0.5 | 0.5 | 0.5 |
| Casor Oil | | | | |
| Linoleic Acid | 0.2 | 0.2 | 0.2 | 0.2 |
| Phase C | | | | |
| Sodium Hydroxide | (Adjust pH) | | | |
| Phase D | | | | |
| Simulgel 600 | 0.5 | 0.5 | 0.5 | 0.5 |
| Phase E | | | | |
| Squalane | 1.5 | 1.5 | 1.5 | 1.5 |
| Oleth-10 | 0.25 | 0.25 | 0.25 | 0.25 |
| Water | 2.5 | 2.5 | 2.5 | 2.5 |
| Green Tea Extract | 2.0 | 2.0 | 2.0 | 2.0 |
| Retinol | 0.1 | 0.1 | 0.1 | 0.1 |
| BHT | 0.05 | 0.05 | 0.05 | 0.05 |

Topical Lotion

| | % w/w | | | |
|---|---|---|---|---|
| Ingredient | 5a | 5b | 5c | 5d |
| Phase A | | | | |
| Water | q.s. 100% | q.s. 100% | q.s. 100% | q.s. 100% |
| Carbomer | 0.2 | 0.2 | 0.2 | 0.2 |
| Glycerin | 2.0 | 2.0 | 2.0 | 2.0 |
| Butylene Glycol | 2.0 | — | 2.0 | — |
| Pentylene Glycol | — | 2.0 | — | 2.0 |
| Sucrose | 0.5 | 0.5 | 0.5 | 0.5 |
| Glucose | 0.5 | 0.5 | 0.5 | 0.5 |
| Sodium Chloride | 0.05 | 0.05 | 0.05 | 0.05 |
| Dehydroaceitc Acid | 0.1 | — | 0.1 | — |
| Chlorhexidine Gluconate | 0.05 | 0.05 | 0.05 | 0.05 |
| Benzalkonium Chloride | — | 0.1 | — | 0.1 |
| Phase B | | | | |
| Cyclomethicone | 5.0 | 5.0 | 5.0 | 5.0 |
| Cetearyl Alcohol | 1.5 | 1.5 | 1.5 | 1.5 |
| Squalane | 2.0 | 2.0 | 2.0 | 2.0 |
| Isostearyl Neopentanoate | 1.0 | 1.0 | 1.0 | 1.0 |
| Cholesterol | 0.5 | 0.5 | 0.5 | 0.5 |
| Caprylic/Capric | 0.5 | 0.5 | 0.5 | 0.5 |
| Triglyceride | | | | |
| Cetearyl Glucoside | 0.2 | 0.2 | 0.2 | 0.2 |
| Linoeic Acid | 0.2 | 0.2 | 0.2 | 0.2 |
| Methyl Glucose | 0.2 | 0.2 | 0.2 | 0.2 |
| Sesquistearate | | | | |
| Cetearyl Glucoside | 0.1 | 0.1 | 0.1 | 0.1 |
| Phenyl Trimethicone | 0.05 | 0.05 | 0.05 | 0.05 |
| TEA-Stearate | 0.1 | 0.1 | 0.1 | 0.1 |
| Phase C | | | | |
| Potasium Hydroxide | (Adjust pH) | | | |
| Phase D | | | | |
| Squalane | 2.0 | 2.0 | 2.0 | 2.0 |
| Retinol | 0.1 | 0.1 | 0.1 | 0.1 |
| Lecithin | 0.1 | 0.1 | 0.1 | 0.1 |
| Water | 4.0 | 4.0 | 4.0 | 4.0 |
| BHA | 0.01 | 0.01 | 0.01 | 0.01 |

Topical Cream

| | % w/w | | | |
|---|---|---|---|---|
| Ingredient | 6a | 6b | 6c | 6d |
| Phase A | | | | |
| Water | q.s. 100% | q.s. 100% | q.s. 100% | q.s. 100% |
| Carbomer | 0.2 | 0.2 | 0.2 | 0.2 |
| Glycerin | 7.0 | 7.0 | 7.0 | 7.0 |
| Dipropylene Glycol | 1.0 | 1.0 | 1.0 | 1.0 |
| PEG-6 | 0.2 | 0.2 | 0.2 | 0.2 |
| PEG-32 | 0.2 | 0.2 | 0.2 | 0.2 |
| JMac | 0.2 | — | 0.2 | — |
| Sodium Benzoate | — | 0.2 | — | 0.2 |
| Dehydroacetic Acid | — | 0.1 | — | 0.1 |
| Phase B | | | | |
| Squalane | 4.5 | 4.5 | 4.5 | 4.5 |
| Pentaerythrityl | 1.0 | 1.0 | 1.0 | 1.0 |
| Tetraoctanoate | | | | |
| Petrolatum | 1.0 | 1.0 | 1.0 | 1.0 |
| Mineral Oil | 1.0 | 1.0 | 1.0 | 1.0 |
| Cyclomethicone | 0.8 | 0.8 | 0.8 | 0.8 |
| Behenyl Alcohol | 0.75 | 0.75 | 0.75 | 0.75 |
| Glyceryl Stearate | 0.5 | 0.5 | 0.5 | 0.5 |
| Stearic Acid | 0.2 | 0.2 | 0.2 | 0.2 |
| PEG-5 Glyceryl Stearate | 0.1 | 0.1 | 0.1 | 0.1 |
| Stearic Acid | 0.1 | 0.1 | 0.1 | 0.1 |
| Potassium Stearate | 0.05 | 0.05 | 0.05 | 0.05 |
| Potassium Isostearate | 0.05 | 0.05 | 0.05 | 0.05 |
| Potassium Behenate | 0.05 | 0.05 | 0.05 | 0.05 |
| Postassium Ascorbyl | 0.05 | 0.05 | 0.05 | 0.05 |
| Tocopheryl Phosphate | | | | |
| Phase C | | | | |
| Potasium Hydroxide | (Adjust pH) | | | |
| Phase D | | | | |
| Squalane | 1.5 | 1.5 | 1.5 | 1.5 |
| Retinyl Palmitate | 0.1 | — | 0.1 | — |
| Retinyl Acetate | — | 0.1 | — | 0.1 |
| Polysorbate-20 | 0.1 | 0.1 | 0.1 | 0.1 |
| Water | 4.0 | 4.0 | 4.0 | 4.0 |
| BHA | 0.02 | 0.02 | 0.02 | 0.02 |

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A topical personal care composition having improved stability of a retinoid, comprising:
a) a retinoid;
b) a preservative selected from the group consisting of phenols, phenol salts, quaternium ammonium compounds, halogens, halogen salts, alcohols, inorganic salts, zinc pyrithione, emulsifying preservatives, and mixtures thereof; and
c) a dermatologically acceptable carrier;
d) wherein the composition is substantially free of parahydroxybenzoic acid esters.

2. A topical composition according to claim 1 wherein the composition comprises from about 0.01% to about 0.5%, by weight of the composition, of the retinoid.

3. A topical composition according to claim 1 wherein the retinoid is selected from the group consisting of retinol, retinol esters, retinal, retinoic acid, and mixtures thereof.

4. A topical composition according to claim 3 wherein the retinoid is selected from the group consisting of retinyl palmitate, retinyl acetate, retinyl propionate, and mixtures thereof.

5. A topical composition according to claim 1 wherein the composition comprises from about 0.001 to about 0.25%, by weight of the composition, of the preservative.

6. A topical composition according to claim 1 wherein the preservative is selected from the group consisting of quaternium ammonium compounds, phenols, phenol salts, and mixtures thereof.

7. A topical composition according to claim 1 wherein the preservative is selected from the group consisting of pentylene glycol, o-phenylphenol, sodium o-phenylphenol, chlorocresol, salicylic acid, salicylic acid salts, chloroxylenol, thymol, triclosan, cresols, benzalkonium chloride, benzethonium chloride, cetylpyridium chloride, sodium dehydroacetate, chlorhexidine, chlorhexidine salts, chloramine T, triclocarban, iodopropynyl butylcarbanate, chlorobutanol, chlorphenesin, hinokitol, silver chloride, zinc pyrithione, alkyldiaminothylglycine hydrochloride, glycerol caprylate, and mixtures thereof.

8. A topical composition according to claim 7 wherein the preservative is selected from the group consisting of pentylene glycol, o-phenylphenol, thymol, benzalkonium chloride, sodium dehydroacetate, chlorhexidine, chlorhexidine gluconate, chloramine T, iodopropynyl butylcarbanate, and mixtures thereof.

9. A topical composition according to claim 1 wherein the composition is substantially free of formaldehyde and substantially free of formaldehyde donating materials.

10. A topical composition according to claim 1 wherein the composition comprises less than 0.01% of parahydroxybenzoic acid esters.

11. A topical composition according to claim 1 wherein the composition further comprises from about 0.01% to about 5% of a preservative enhancer selected from the group consisting of propylene glycol, butylene glycol, EDTA, disodium EDTA, tetrasodium EDTA, and mixtures thereof.

12. A topical composition according to claim 1 wherein the composition further comprises a skin care active selected from the group consisting of vitamins, proteins, zeolites, peptides, skin-lightening agents, sunscreen actives, terpene alcohols, desquamation actives, anti-acne actives, anti-wrinkle actives, anti-atrophy actives, anti-oxidants, flavanoids, anti-inflammatory agents, anti-cellulite agents, topical anesthetics, tanning actives, skin soothing actives, skin healing actives, conditioning agents, and mixtures thereof.

13. A method of regulating the condition of skin, said method comprising applying to the skin of a human in need of treatment, a safe and effective amount of a composition according to claim 1.

14. A topical personal care composition having improved stability of a retinoid, comprising:
a) a retinoid selected from the group consisting of retinyl esters, retinyl aldehydes, and mixtures thereof;
b) a preservative; and
c) a dermatologically acceptable carrier;
d) wherein the composition is substantially free of parahydroxybenzoic acid esters; and
e) wherein the composition is substantially free of formaldehyde and formaldehyde donating compounds.

15. A topical composition according to claim 14 wherein the composition comprises from about 0.01% to about 0.5%, by weight of the composition, of the retinoid.

16. A topical composition according to claim 15 wherein the retinoid is selected from the group consisting of retinyl palmitate, retinyl propionate, retinyl acetate, and mixtures thereof.

17. A topical composition according to claim 16 wherein the retinoid is retinyl propionate.

18. A topical composition according to claim 14 wherein the composition comprises from about 0.001 to about 0.25%, by weight of the composition, of the preservative.

19. A topical composition according to claim 14 wherein the preservative is selected from the group consisting of phenols, phenol salts, carboxylic acids, carboxylic acid salts, quaternium ammonium compounds, halogens, halogen salts, alcohols, inorganic salts, heterocyclic compounds, emulsifying preservatives, and mixtures thereof.

20. A topical composition according to claim 19 wherein the preservative is selected from the group consisting of pentylene glycol, o-phenylphenol, sodium o-phenylphenol, chlorocresol, salicylic acid, sodium salicylate, magnesium salicylate, resorcin, chloroxylenol, thymol, triclosan, cresols, benzalkonium chloride, benzethonium chloride, cetylpyridium chloride, benzoic acid, sodium benzoate, sorbic acid, dehydroacetic acid, sodium dehydroacetate, chlorhexidine, chlorhexidine gluconate, chloramine T, triclocarban, iodopropynyl butylcarbanate, chlorobutanol, chlorphenesin, hinokitol, silver chloride, zinc pyrithione, alkyldiaminothylglycine hydrochloride, glycerol caprylate, and mixtures thereof.

21. A topical composition according to claim 20 wherein the preservative is selected from the group consisting of pentylene glycol, o-phenylphenol, thymol, benzalkonium chloride, dehydroacetic acid, sodium dehydroacetate, chlorhexidine, chlorhexidine gluconate, chloramine T, iodopropynyl butylcarbanate, and mixtures thereof.

22. A topical composition according to claim 14 wherein the composition comprises less than 0.01% of parahydroxybenzoic acid esters.

23. A topical composition according to claim 14 wherein the composition further comprises from about 0.01% to about 5% of a preservative enhancer selected from the group consisting of pentylene glycol, butylene glycol, EDTA, disodium EDTA, tetrasodium EDTA, and mixtures thereof.

24. A topical composition according to claim 14 wherein the composition further comprises a skin care active selected from the group consisting of vitamins, proteins, zeolites, peptides, skin-lightening agents, sunscreen actives, terpene alcohols, desquamation actives, anti-acne actives, anti-wrinkle actives, anti-atrophy actives, anti-oxidants, flavanoids, anti-inflammatory agents, anti-cellulite agents, topical anesthetics, tanning actives, skin soothing actives, skin healing actives, conditioning agents, and mixtures thereof.

25. A method of regulating the condition of skin and/or hair, said method comprising applying to the skin and/or hair of a human in need of treatment, a safe and effective amount of a composition according to claim 14.

26. A topical personal care composition having improved stability of a retinoid, comprising:
a) from about 0.0001% to about 2% of a retinoid;
b) an anti-oxidant;
c) from about 0.1% to about 0.35% of a preservative selected from the group consisting of carboxylic acid, carboxylic acid salts, and mixtures thereof; and
d) a dermatologically acceptable carrier;
e) wherein the composition is substantially free of parahydroxybenzoic acid esters; and
f) wherein the composition is substantially free of formaldehyde and formaldehyde donating compounds.
